SEQUENCE LISTING (8 sequences)

SEQ ID NO: 1 - DNA, 430 bp, Homo sapiens
tgtgattgaagacgatggcctccacgcagccttcactgtggctcacgaaatcggacatttacttggcctctcccatgacgattccaaattctgtgaagagacctttggttccacagaagataagcgcttaatgtcttccatccttaccagcattgatgcatctaagccctggtccaaatgcacttcagccaccatcacagaattcctggatgatggccatggtaactgtttgctggacctaccacgaaagcagatcctgggccccgaagaactcccaggacagacctacgatgccacccagcagtgcaacctgacattcgggcctgagtactccgtgtgtcccggcatggatgtctgtgctcgcctgtggtgtgctgtggtacgccagggccagatggtctgtctgaccaagaagctgcctgcggtggaa

SEQ ID NO: 2 - DNA, 20 bp, Artificial Sequence
  primer
ctaagccctggtccaaatgc

SEQ ID NO: 3 - DNA, 21 bp, Artificial Sequence
  primer
ccagcaaacagttaccatggc

SEQ ID NO: 4 - DNA, 27 bp, Artificial Sequence
  probe
ttcagccaccatcacagaattcctgga

SEQ ID NO: 5 - DNA, 467 bp, Homo sapiens
cgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatg

SEQ ID NO: 6 - DNA, 20 bp, Artificial Sequence
  primer
ccagctcaccatggatgatg

SEQ ID NO: 7 - DNA, 17 bp, Artificial Sequence
  primer
ccttgcacatgccggag

SEQ ID NO: 8 - DNA, 20 bp, Artificial Sequence
  probe
ccgcgctcgtcgtcgacaac

(19) United States
(12) Patent Application Publication
(10) Pub. No.: US 2019/0185912 A1
(43) Pub. Date: Jun. 20, 2019

(54) ADAMTS5 Gene Quantification Method, Primer Pair, Combination of Primer Pair and Probe, and Kit

(71) Applicant: KayteeBio Co. & Ltd., Funabashi-shi, Chiba (JP)

(72) Inventor: Kensei TSUZAKA, Funabashi-shi, Chiba (JP)

(21) Appl. No.: 16/301,091

(22) PCT Filed: May 20, 2016
(86) PCT No.: PCT/JP2016/064983

§ 371 Date: Nov. 13, 2018

(51) Int. Cl. C12Q 1/686; C12Q 1/6851; C12Q 1/6858

(52) U.S. Cl. CPC C12Q 1/686; C12Q 1/6851; C12Q 2600/16; C12Q 2600/166; C12Q 1/6858

(57) ABSTRACT

A method for quantifying an ADAMTS5 gene, the method includes: measuring an amount of the ADAMTS5 gene in a sample through a quantitative PCR, wherein a primer consisting of a nucleotide sequence set forth in SEQ ID NO: 2 and a primer consisting of a nucleotide sequence set forth in SEQ ID NO: 3 are used as an amplification primer pair for amplifying a region including the ADAMTS5 gene.

### TECHNICAL FIELD

The present invention relates to a method for quantifying an ADAMTS5 gene in a sample through a quantitative PCR, a primer pair and a combination of a primer pair and a probe used for quantifying an ADAMTS5 gene in a sample through a quantitative PCR, and a kit used for quantifying an ADAMTS5 gene in a sample through a quantitative PCR.

### BACKGROUND ART

ADAMTS (a disintegrin and metalloproteinase with thrombospondin motifs) 5 is an enzyme that belongs to the ADAMTS family and is involved in cartilage destruction.

The amount of the ADAMTS5 is known to be an indicator for evaluating whether an anti-TNFα antibody drug is effective to rheumatoid arthritis (see, for example, Patent Literatures 1 and 2).

Generally, quantification of an mRNA of a gene is performed by using the real-time PCR. When an mRNA of the ADAMTS5 gene is quantified, its quantity is often represented by a ratio of the amount of the mRNA of the ADAMTS5 gene to the amount of an mRNA of a control gene, such as an mRNA of a β-actin gene (hereinafter may be referred to as a “relative ratio”).

In recent years, as one technique of the real-time PCR, a technique using a TaqMan (registered trademark) MGB probe (available from Thermo Fisher Scientific K.K.) has been widely used. The MGB probe is advantageous in that a probe having a Tm value of 70° C. can be designed so as to have a length of 20 or less bases due to MGB (Minor Groove Binder) structure that is a Tm enhancer.

However, use of the MGB probe is costly, which is problematic.

In addition, as described above, the amount of the ADAMTS5 gene is an indicator for predicting drug efficacy. Therefore, there is a need to develop a method capable of more highly sensitively measuring the amount of the ADAMTS5 gene.

Therefore, a method capable of performing quantification of the ADAMTS5 gene in the sample at low cost and in a highly sensitive manner has not been provided yet. Then, there is a strong need to rapidly provide such a method.

### CITATION LIST

### Patent Literature

PTL 1 International Publication No. WO 2010/038840

PTL 2 Japanese Patent Application Laid-Open No. 2011-109929

### SUMMARY OF INVENTION

### Technical Problem

The present invention can solve the above existing problems and can achieve the following object. That is, an object of the present invention is to provide a method for quantifying an ADAMTS5 gene that can perform quantification of the ADAMTS5 gene in a sample at low cost and in a highly sensitive manner, a primer pair, a combination of a primer pair and a probe, and a kit that can be suitably used for the method for quantifying the ADAMTS5 gene.

### Solution to Problem

Means for solving the problems are as follows. That is,

<1> A method for quantifying an ADAMTS5 gene, the method including:

measuring an amount of the ADAMTS5 gene in a sample through a quantitative PCR,

wherein a primer consisting of a nucleotide sequence set forth in SEQ ID NO: 2 below and a primer consisting of a nucleotide sequence set forth in SEQ ID NO: 3 below are used as an amplification primer pair for amplifying a region including the ADAMTS5 gene,

(SEQ ID NO: 2)
5′-CTAAGCCCTGGTCCAAATGC-3′;
and
(SEQ ID NO: 3)
5′-CCAGCAAACAGTTACCATGGC-3′.

<2> A primer pair used for quantifying an ADAMTS5 gene in a sample through a quantitative PCR, the primer pair consisting of

a primer consisting of a nucleotide sequence set forth in SEQ ID NO: 2 below; and

a primer consisting of a nucleotide sequence set forth in SEQ ID NO: 3 below,

(SEQ ID NO: 2)
5′-CTAAGCCCTGGTCCAAATGC-3′;
and
(SEQ ID NO: 3)
5′-CCAGCAAACAGTTACCATGGC-3′.

<3> A combination of a primer pair and a probe used for quantifying an ADAMTS5 gene in a sample through a quantitative PCR,

wherein the primer pair is the primer pair according to <2>, and

wherein the probe is a probe consisting of a nucleotide sequence set forth in SEQ ID NO: 4 below:

(SEQ ID NO: 4)
5′-TTCAGCCACCATCACAGAATTCCTGGA-3′.

<4> A kit used for quantifying an ADAMTS5 gene in a sample through a quantitative PCR, the kit including:

the primer pair according to <2>.

### Advantageous Effects of Invention

According to the present invention, it is possible to solve the above existing problems, achieve the above object, and provide a method for quantifying an ADAMTS5 gene that can perform quantification of the ADAMTS5 gene in a sample at low cost and in a highly sensitive manner, a primer pair, a combination of a primer pair and a probe, and a kit that can be suitably used for the method for quantifying the ADAMTS5 gene.

### BRIEF DESCRIPTION OF DRAWINGS

FIG. 1A is a graph presenting an amplification curve obtained through a real-time PCR in Test Example 1-1.

FIG. 1B is a graph presenting an amplification curve obtained through a real-time PCR in Test Example 1-3.

FIG. 2A is a graph presenting an amplification curve obtained through a real-time PCR in Test Example 2-1.

FIG. 2B is a graph presenting an amplification curve obtained through a real-time PCR in Test Example 2-3.

### DESCRIPTION OF EMBODIMENTS

### (Method for Quantifying ADAMTS5 Gene)

A method for quantifying an ADAMTS5 gene of the present invention includes at least a step of measuring the amount of an ADAMTS5 gene (hereinafter may be referred to as “ADAMTS5 gene amount measuring step”), and further includes other steps if necessary.

### <ADAMTS5 Gene Amount Measuring Step>

The ADAMTS5 gene amount measuring step is a step of measuring an amount of the ADAMTS5 gene in a sample through a quantitative PCR.

In the ADAMTS5 Gene Amount Measuring Step, the Amount of mRNA of the ADAMTS5 gene in the sample is quantified.

### <<Sample>>

The sample is not particularly limited and may be appropriately selected depending on the intended purpose. Examples thereof include peripheral blood, a synovial membrane, synovial fluid, samples obtained by extracting RNAs from the aforementioned samples, and samples obtained by preparing cDNAs from the RNAs. Among them, peripheral blood, samples obtained by extracting RNAs from the peripheral blood, and samples obtained by preparing cDNAs from the RNAs are preferable because they are conveniently taken.

What the sample is derived from is not particularly limited and may be appropriately selected depending on the intended purpose, but human is preferable.

A method for preparing the peripheral blood, the synovial membrane, and the synovial fluid is not particularly limited and may be appropriately selected from known methods.

A method for extracting RNAs from, for example, the peripheral blood is not particularly limited and may be appropriately selected from known methods.

A method for preparing a cDNA from the RNA is not particularly limited and may be appropriately selected from known methods.

### <<ADAMTS5 Gene>>

The ADAMTS (a disintegrin and metalloproteinase with thrombospondin motifs) 5 is an enzyme that belongs to the ADAMTS family and is involved in cartilage destruction.

A nucleotide sequence of the ADAMTS5 gene is known. The nucleotide sequence thereof is easily available from public databases such as GenBank (NCBI). For example, the nucleotide sequence of human ADAMTS5 gene is available under NCBI accession number NM_007038.3.

### <<Quantitative PCR>>

The quantitative PCR is performed using an amplification primer pair of the present invention for amplifying a region including the ADAMTS5 gene (hereinafter this amplification primer pair may be referred to as “primer pair for ADAMTS5 gene”).

### —Primer Pair for ADAMTS5 Gene—

The primer pair for the ADAMTS5 gene consists of a forward primer consisting of a nucleotide sequence set forth in SEQ ID NO: 2 below and a reverse primer consisting of a nucleotide sequence set forth in SEQ ID NO: 3 below.

(SEQ ID NO: 2)
5′-CTAAGCCCTGGTCCAAATGC-3
(SEQ ID NO: 3)
5′-CCAGCAAACAGTTACCATGGC-3′

Note that, the nucleotide sequence set forth in SEQ ID NO: 3 corresponds to bases at from 2,149th to 2,129th of NCBI accession number NM_007038.3 and corresponds to a part of exon 3 and a part of exon 4. In the nucleotide sequence set forth in SEQ ID NO: 3, the “CCAGCAAACAGTTAC” at a side of the 5′ terminal corresponds to the exon 3 and the “CATGGC” at a side of the 3′ terminal corresponds to the exon 4.

A method for producing the primer pair for the ADAMTS5 gene is not particularly limited and may be appropriately selected depending on the intended purpose. The primer pair for the ADAMTS5 gene can be produced, for example, through chemical synthesis.

### —Detection Probe for ADAMTS5 Gene—

A detection probe for the ADAMTS5 gene is not particularly limited and may be appropriately selected depending on the intended purpose, but a probe constituting of a nucleotide sequence set forth in SEQ ID NO: 4 below is preferable.

(SEQ ID NO: 4)
5′-TTCAGCCACCATCACAGAATTCCTGGA-3′

The detection probe for the ADAMTS5 gene preferably has a 5′ terminal modified with a reporter dye and a 3′ terminal modified with a quencher dye. A combination of the reporter dye and the quencher dye is not particularly limited and may be appropriately selected depending on the intended purpose. Examples thereof include a combination of FAM and TAMRA, a combination of HEX and TAMRA, a combination of TET and TAMRA, a combination of FAM and BHQ1, a combination of HEX and BHQ1, a combination of TET and BHQ1, a combination of TAMRA and BHQ2, and a combination of Cyanine5 and BHQ3.

A method for producing the detection probe for the ADAMTS5 gene is not particularly limited and may be appropriately selected depending on the intended purpose. The detection probe for the ADAMTS5 gene can be produced, for example, through chemical synthesis.

### —Quantitative PCR—

The quantitative PCR is not particularly limited and may be appropriately selected depending on the intended purpose, so long as the primer pair for the ADAMTS5 gene is used. A real-time PCR using a fluorescent probe is preferable, and a real-time PCR using the detection probe for the ADAMTS5 gene is more preferable.

In the quantitative PCR, it is possible to quantify at least one of an absolute amount of the ADAMTS5 gene and a relative amount with respect to the amount of the below-described control gene (the amount of the ADAMTS5 gene/the amount of the control gene).

Reaction conditions of the quantitative PCR are not particularly limited and may be appropriately selected depending on the intended purpose.

For example, a reaction reagent used in the real-time PCR is not particularly limited and may be appropriately selected from known reagents depending on the intended purpose. Use of Premix Ex Taq (Probe qPCR) available from Takara Bio Inc. is preferable.

The amount of a template DNA used in the real-time PCR is not particularly limited and may be appropriately selected depending on the intended purpose.

The amount of the forward primer used in the real-time PCR is not particularly limited and may be appropriately selected depending on the intended purpose, but the final concentration thereof is preferably about 900 nM.

The amount of the reverse primer used in the real-time PCR is not particularly limited and may be appropriately selected depending on the intended purpose, but the final concentration thereof is preferably about 300 nM.

The amount of the probe used in the real-time PCR is not particularly limited and may be appropriately selected depending on the intended purpose, but the final concentration thereof is preferably about 75 nM.

A reaction time of the real-time PCR is not particularly limited and may be appropriately selected depending on the intended purpose. However, the following cycle: (1) 95° C. for 30 seconds; and thereafter (2) 95° C. for 15 seconds, then 60° C. for 60 seconds is preferably repeated a plurality of times.

The number of the cycles of the (2) 95° C. for 15 seconds, then 60° C. for 60 seconds is not particularly limited and may be appropriately selected depending on the intended purpose, but is preferably about 50 cycles.

The quantitative method using the real-time PCR is not particularly limited and may be appropriately selected depending on the intended purpose. Examples thereof include a calibration curve method.

The calibration curve method is a method for preparing a calibration curve and then quantifying the sample using the calibration curve.

A DNA used as a template of the control DNA in the calibration curve method is not particularly limited and may be appropriately selected depending on the intended purpose, so long as a concentration thereof is known. Examples thereof include the whole cDNA of the ADAMTS5 obtained from healthy human and if necessary purified, and a plasmid into which the ADAMTS5 cDNA has been incorporated.

Regarding the real-time PCR, a reverse transcription reaction may be performed in a device of the real-time PCR, or a cDNA that has been prepared in advance may be used.

A device used in the quantitative PCR is not particularly limited and may be appropriately selected from known devices depending on the intended purpose.

### <Other Steps>

The other steps are not particularly limited and may be appropriately selected depending on the intended purpose, so long as the other steps do not degrade the effect of the present invention. Examples thereof include a step of measuring the amount of a control gene (which may be referred to as “gene used as an internal standard”) (hereinafter this step may be referred to as “control gene amount measuring step”).

### <<Control Gene Amount Measuring Step>>

The control gene amount measuring step is a step of measuring the amount of the control gene in a sample through the quantitative PCR.

In the control gene amount measuring step, the amount of the mRNA of the control gene in the sample is quantified.

### —Sample—

The sample is the same sample as that of the <<Sample>> in the section of the <ADAMTS5 gene amount measuring step> as described above.

### —Control Gene—

The control gene is not particularly limited and may be appropriately selected to depending on the intended purpose. Examples thereof include a β-actin gene and a GAPDH (glyceraldehyde-3-phosphate dehydrogenase) gene. Among them, a β-actin gene is preferable.

### —β-Actin Gene—

A nucleotide sequence of the β-actin gene is known and the nucleotide sequence thereof is easily available from public databases such as GenBank (NCBI). For example, the nucleotide sequence of human β-actin gene is available under NCBI accession number NM_001101.2.

### —Quantitative PCR—

Hereinafter, a quantitative PCR using the J-actin gene as the control gene will be described.

A primer pair used in the quantitative PCR of the β-actin gene is not particularly limited and may be appropriately selected depending on the intended purpose. The following amplification primer pair for amplifying a region including the β-actin gene (hereinafter this primer pair may be referred to as “primer pair for β-actin gene”) is preferable.

A probe used in the quantitative PCR of the β-actin gene is not particularly limited and may be appropriately selected depending on the intended purpose, but the following detection probe for the β-actin gene is preferable.

A combination of a primer pair and a probe used in the quantitative PCR of the β-actin gene is not particularly limited and may be appropriately selected depending on the intended purpose, but a combination of the following primer pair for the β-actin gene and the following detection probe for the β-actin gene is preferable.

—Primer pair for β-actin gene—

The primer pair for the β-actin gene consists of a forward primer consisting of a nucleotide sequence set forth in SEQ ID NO: 6 below and a reverse primer consisting of a nucleotide sequence set forth in SEQ ID NO: 7 below.

(SEQ ID NO: 6)
5′-CCAGCTCACCATGGATGATG-3′
(SEQ ID NO: 7)
5′-CCTTGCACATGCCGGAG-3′

Note that, the nucleotide sequence set forth in SEQ ID NO: 6 corresponds to bases at from 64th to 83rd of NCBI accession number NM_001101.2 and corresponds to a part of the exon 1 and a part of the exon 2. In the nucleotide sequence set forth in SEQ ID NO: 6, the “CCAG” at a side of the 5′ terminal corresponds to the exon 1 and the “CTCACCATGGATGATG” at a side of the 3′ terminal corresponds to the exon 2.

A method for producing the primer pair for the β-actin gene is not particularly limited and may be appropriately selected depending on the intended purpose. The primer pair for the β-actin gene can be produced, for example, through chemical synthesis.

### —Detection Probe for β-Actin Gene—

The detection probe for the β-actin gene consists of a nucleotide sequence set forth in SEQ ID NO: 8 below.

(SEQ ID NO: 8)
5′-CCGCGCTCGTCGTCGACAAC-3′

The detection probe for the β-actin gene preferably has a 5′ terminal modified with a reporter dye and a 3′ terminal modified with a quencher dye.

A combination of the reporter dye and the quencher dye is the same combination as that described in the section of the —Detection probe for the ADAMTS5 gene— of the <ADAMTS5 gene amount measuring step> as described above.

A method for producing the detection probe for the β-actin gene is not particularly limited and may be appropriately selected depending on the intended purpose. The detection probe for the β-actin gene can be produced, for example, through chemical synthesis.

### —Quantitative PCR—

A quantitative PCR of the β-actin gene is not particularly limited and may be appropriately selected depending on the intended purpose, but a real-time PCR using a fluorescent probe is preferable.

The quantitative PCR of the β-actin gene can quantify an absolute amount of the β-actin gene. In addition, it can be used for quantification of a relative amount of the above-described ADAMTS5 gene in the sample.

Reaction conditions of the quantitative PCR of the β-actin gene are not particularly limited and may be appropriately selected depending on the intended purpose.

For example, a reaction reagent used in the real-time PCR of the β-actin gene is not particularly limited and may be appropriately selected from known reagents depending on the intended purpose. Use of Premix Ex Taq (Probe qPCR) available from Takara Bio Inc. is preferable.

The amount of the template DNA used in the real-time PCR of the β-actin gene is not particularly limited and may be appropriately selected depending on the intended purpose.

The amount of the forward primer used in the real-time PCR of the β-actin gene is not particularly limited and may be appropriately selected depending on the intended purpose, but the final concentration thereof is preferably about 900 nM.

The amount of the reverse primer used in the real-time PCR of the β-actin gene is not particularly limited and may be appropriately selected depending on the intended purpose, but the final concentration thereof is preferably about 300 nM.

The amount of the probe in the real-time PCR of the β-actin gene is not particularly limited and may be appropriately selected depending on the intended purpose, but the final concentration thereof is preferably about 50 nM.

A reaction time of the real-time PCR of the β-actin gene is not particularly limited and may be appropriately selected depending on the intended purpose. However, the following cycle: (1) 95° C. for 30 seconds; and thereafter (2) 95° C. for 15 seconds, then 60° C. for 60 seconds is preferably repeated a plurality of times.

The number of the cycles of the (2) 95° C. for 15 seconds, then 60° C. for 60 seconds is not particularly limited and may be appropriately selected depending on the intended purpose, but is preferably about 50 cycles.

A method for quantifying the β-actin gene through the real-time PCR is the same method as that described in the section of the —Quantitative PCR— of the <ADAMTS5 gene amount measuring step> as described above.

Regarding the real-time PCR of the β-actin gene, a reverse transcription reaction may be performed in a device of the real-time PCR, or a cDNA that has been prepared in advance may be used.

A device used in the quantitative PCR of the β-actin gene is not particularly limited and may be appropriately selected from known devices depending on the intended purpose.

### (Primer Pair)

A primer pair of the present invention is a primer pair used for quantifying the ADAMTS5 gene in the sample through the quantitative PCR.

<Primer pair>

The primer pair is the same primer pair as that described in the section of the —Primer pair for ADAMTS5 gene— of the <ADAMTS5 gene amount measuring step> of the (Method for quantifying ADAMTS5 gene) as described above.

### (Combination of Primer Pair and Probe)

A combination of the primer pair and the probe of the present invention is a combination of the primer pair and the probe used for quantifying the ADAMTS5 gene in the sample through the quantitative PCR.

### <Primer Pair>

The primer pair is the same as that described in the section of the —Primer pair for ADAMTS5 gene— of the <ADAMTS5 gene amount measuring step> of the (Method for quantifying ADAMTS5 gene) as described above.

### <Probe>

The probe is the same as that described in the section of the —Detection probe for the ADAMTS5 gene— of the <ADAMTS5 gene amount measuring step> of the (Method for quantifying ADAMTS5 gene) as described above.

### (Kit)

A kit of the present invention is a kit used for quantifying the ADAMTS5 gene in the sample through the quantitative PCR. The kit of the present invention includes at least the primer pair of the present invention, and further includes other elements such as a probe if necessary.

### <Primer Pair of the Present Invention>

The primer pair is the primer pair for the ADAMTS5 gene of the present invention as described above.

### <Other Elements>

The other elements are not particularly limited and may be appropriately selected depending on the intended purpose, so long as they do not degrade the effect of the present invention. Examples thereof include a detection probe for the ADAMTS5 gene, an amplification primer pair for amplifying a region including a control gene (hereinafter this primer pair may be referred to as “primer pair for control gene”), a detection probe for a control gene, and a reagent used for PCR.

### <<Detection Probe for ADAMTS5 Gene>>

The probe is the same probe as that described in the section of the —Detection probe for ADAMTS5 gene— of the <ADAMTS5 gene amount measuring step> of the (Method for quantifying ADAMTS5 gene) as described above.

### <<Primer Pair for Control Gene and Detection Probe for Control Gene>>

The control gene is the same control gene as those described in the section of the —Control gene— of the <<Control gene amount measuring step>> of the <Other steps> of the (Method for quantifying ADAMTS5 gene) as described above. A preferable gene is also the same.

The kit preferably includes, as the other elements, the detection probe for the ADAMTS5 gene, more preferably includes the detection probe for the ADAMTS5 gene and the primer pair for the β-actin gene, preferably includes the detection probe for the ADAMTS5 gene, the primer pair for the β-actin gene, and the detection probe for the β-actin gene.

The primer pair for the β-actin gene and the detection probe for the β-actin gene are the same as those described in the section of the —Primer pair for β-actin gene— and the —Detection probe for β-actin gene— of the <<Control gene amount measuring step>> of the <Other steps> of the (Method for quantifying ADAMTS5 gene) as described above.

### EXAMPLES

The present invention will be described in more detail by way of the following Preparation Examples and Test Examples. However, the present invention should not be construed as being limited to, for example, these Test Examples.

### Preparation Example 1: Preparation of cDNA of ADAMTS5 Gene

A DNA consisting of a nucleotide sequence set forth in SEQ ID NO: 1 was inserted into a plasmid and then was subjected to a restriction enzyme treatment to prepare a cDNA of the ADAMTS5 gene. The nucleotide sequence set forth in SEQ ID NO: 1 corresponds to bases at from 1,914th to 2,343th (430 bp) of ADAMTS5 (NCBI Reference Sequence: NM_007038.3).

### Preparation Example 2: Preparation of Primer Pair and Probe for ADAMTS5 Gene

The following amplification primer pair for amplifying a region including the ADAMTS5 gene and the following detection probe for the ADAMTS5 gene were prepared through chemical synthesis.

Forward Primer

(SEQ ID NO: 2)
5′-CTAAGCCCTGGTCCAAATGC-3′

Reverse Primer

(SEQ ID NO: 3)
5′-CCAGCAAACAGTTACCATGGC-3′

Probe

(SEQ ID NO: 4)
5′-FAM-TTCAGCCACCATCACAGAATTCCTGGA-TAMRA-3′

The probe set forth in the above SEQ ID NO: 4 has a 5′ terminal modified with FAM (reporter dye) and a 3′ terminal modified with TAMRA (quencher dye).

### Preparation Example 3: Preparation of cDNA of β-Actin Gene

A DNA consisting of a nucleotide sequence set forth in SEQ ID NO: 5 was inserted into a plasmid and then was subjected to a restriction enzyme treatment to prepare a cDNA of the β-actin gene. The nucleotide sequence set forth in SEQ ID NO: 5 corresponds to bases at from 3rd to 469th (467 bp) of β-actin (NCBI Reference Sequence: NM_001101.2).

### Preparation Example 4: Preparation of Primer Pair for β-Actin Gene and Probe

The following amplification primer pair for amplifying a region including the β-actin gene and the following detection probe for the β-actin gene were prepared through chemical synthesis.

Forward Primer

(SEQ ID NO: 6)
5′-CCAGCTCACCATGGATGATG-3′

Reverse Primer

(SEQ ID NO: 7)
5′-CCTTGCACATGCCG-GAG-3′

Probe

(SEQ ID NO: 8)
5′-FAM-CCGCGCTCGTCGTCGACAAC-TAMRA-3′

The probe set forth in the SEQ ID NO: 8 has a 5′ terminal modified with FAM (reporter dye) and a 3′ terminal modified with TAMRA (quencher dye).

### Test Example 1: Quantification Through Real-Time PCR

The primer pair and the probe of the present invention were used to perform the real-time PCR as described in the following Test Examples 1-1 to 1-4.

### Test Example 1-1

The cDNA of the ADAMTS5 gene prepared in the Preparation Example 1 was diluted so as to be 1.0 μg/mL, 1.0×10⁻³ μg/mL, or 1.0×10⁻⁶ μg/mL to be used as a control DNA.

As a primer pair and a probe, the primer pair for the ADAMTS5 gene (SEQ ID NOs: 2 and 3) and the probe (SEQ ID NO: 4) prepared in the Preparation Example 2 were used.

The real-time PCR was performed using ABI PRISM 7000 (available from Thermo Fisher Scientific K.K.) and reaction reagents described in Table 1-1 under the below-described reaction conditions of the reaction time.

TABLE 1-1
Reaction reagents

| | | Final |
|---|---|---|
| | Amount | concentration |
| Premix Ex Taq (Probe qPCR) (2×) | 15 μL | 1× |
| (available from Takara Bio Inc.) | | |
| Forward primer (45 μM) | 0.6 μL | 900 nM |
| Reverse primer (15 μM) | 0.6 μL | 300 nM |
| Probe (10 μM) | 0.225 μL | 75 nM |
| ROX Reference Dye (50×) | 0.6 μL | 1× |
| (available from Takara Bio Inc.) | | |
| Control DNA | 3.0 μL | |
| Sterile distilled water | 9.975 μL | |
| Total | 30 μL | |

### —Reaction Time—

The real-time PCR was performed in the following order.

(1) 95° C. for 30 seconds

(2) The number of cycles: 50

Conditions: 95° C. for 15 seconds, then 60° C. for 60 seconds

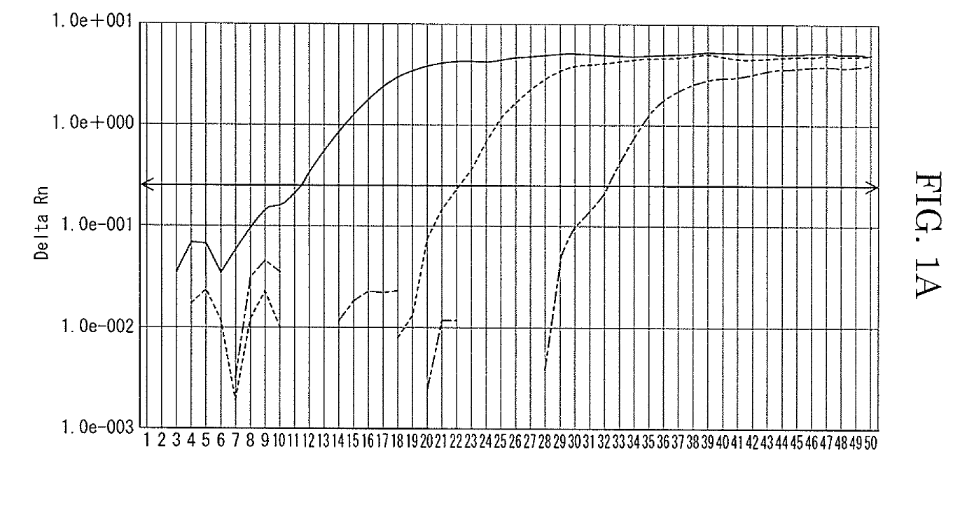

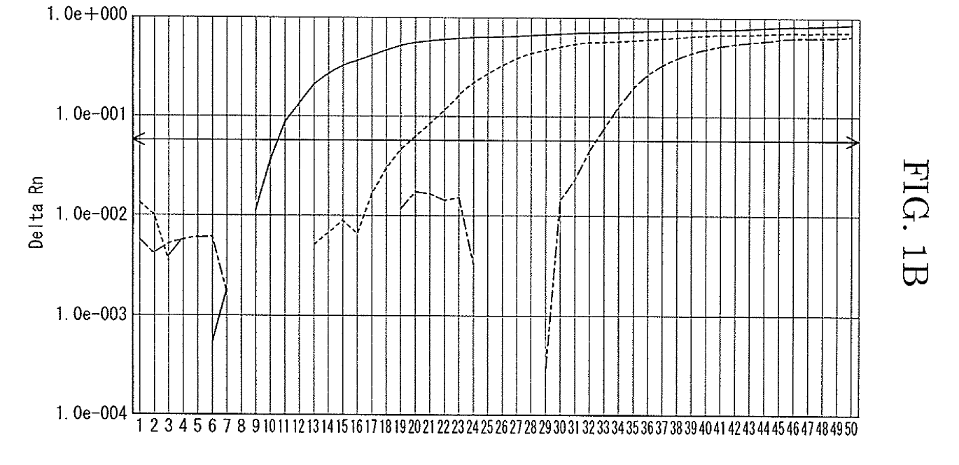

Amplification curves obtained through the real-time PCR are presented in FIG. 1A (horizontal axis: the number of cycles, longitudinal axis: fluorescent signal (ΔRn) corresponding to the amplified PCR products). In FIG. 1A, a solid line presents a result obtained when the control DNA of 1.0 μg/mL was used, a dotted line presents a result obtained when the control DNA of 1.0×10⁻³ μg/mL was used, a dot-dash line presents a result obtained when the control DNA of 1.0×10⁻⁶ μg/mL was used, and a line ends of which are arrows presents a base line (Threshold Line).

From the results of the amplification curves, a calibration curve was prepared with the longitudinal axis presenting a concentration of the control DNA and the horizontal axis presenting a Ct value (the number of cycles corresponding to Threshold).

### Test Example 1-2

Two kinds of healthy human's cDNAs (hereinafter may be referred to as “sample 1” and “sample 2”) were provided and were 2-fold diluted to prepare samples.

As a primer pair and a probe, the primer pair for the ADAMTS5 gene (SEQ ID NOs: 2 and 3) and the probe (SEQ ID NO: 4) prepared in the Preparation Example 2 were used.

The real-time PCR was performed under the same reaction conditions of the real-time PCR as in the Test Example 1-1 except that the control DNA in the Test Example 1-1 was changed to the sample 1 or 2.

When the calibration curve obtained in the Test Example 1-1 was used to calculate the samples 1 and 2 for an absolute amount of the ADAMTS5 gene, the result of the sample 1 was 1.07×10⁻³ μg/mL and the result of the sample 2 was 2.18×10⁻³ μg/mL.

### Test Example 1-3

The cDNA of the β-actin gene prepared in the Preparation Example 3 was diluted so as to be 1.0 μg/mL, 1.0×10⁻³ μg/mL, or 1.0×10⁻⁶ μg/mL to be used as a control DNA.

As a primer pair and a probe, the primer pair for the β-actin gene (SEQ ID NOs: 6 and 7) and the probe (SEQ ID NO: 8) prepared in the Preparation Example 4 were used.

The real-time PCR was performed using ABI PRISM 7000 (available from Thermo Fisher Scientific K.K.) and reaction reagents described in Table 1-2 under the below-described reaction conditions of the reaction time.

TABLE 1-2
Reaction reagents

| | | Final |
|---|---|---|
| | Amount | concentration |
| Premix Ex Taq (Probe qPCR) (2×) | 15 μL | 1× |
| (available from Takara Bio Inc.) | | |
| Forward primer (45 μM) | 0.6 μL | 900 nM |
| Reverse primer (15 μM) | 0.6 μL | 300 nM |
| Probe (10 μM) | 0.15 μL | 50 nM |
| ROX Reference Dye (50×) | 0.6 μL | 1× |
| (available from Takara Bio Inc.) | | |
| Control DNA | 3.0 μL | |
| Sterile distilled water | 10.05 μL | |
| Total | 30 μL | |

### —Reaction Time—

The real-time PCR was performed in the following order.

(1) 95° C. for 30 seconds

(2) The number of cycles: 50

Conditions: 95° C. for 15 seconds, then 60° C. for 60 seconds

Amplification curves obtained through the real-time PCR are presented in FIG. 1B (horizontal axis: the number of cycles, longitudinal axis: fluorescent signal (ΔRn) corresponding to the amplified PCR products). In FIG. 1B, a solid line presents a result obtained when the control DNA of 1.0 μg/mL was used, a dotted line presents a result obtained when the control DNA of 1.0×10⁻³ μg/mL was used, a dot-dash line presents a result obtained when the control DNA of 1.0×10⁻⁶ μg/mL was used, and a line ends of which are arrows presents a base line (Threshold Line).

From the results of the amplification curves, a calibration curve was prepared with the longitudinal axis presenting a concentration of the control DNA and the horizontal axis presenting a Ct value (the number of cycles corresponding to Threshold).

### Test Example 1-4

Two kinds of healthy human's cDNAs (sample 1 and sample 2) were provided and were 50-fold diluted to prepare samples.

As a primer pair and a probe, the primer pair for the β-actin gene (SEQ ID NOs: 6 and 7) and the probe (SEQ ID NO: 8) prepared in the Preparation Example 4 were used.

The real-time PCR was performed under the same reaction conditions of the real-time PCR as in the Test Example 1-3 except that the control DNA in the Test Example 1-3 was changed to the sample 1 or 2

When the calibration curve obtained in the Test Example 1-3 was used to calculate the samples 1 and 2 for an absolute amount of the β-actin gene, the result of the sample 1 was 5.00 μg/mL and the result of the sample 2 was 6.40 μg/mL.

From the results of Test Examples 1-1 to 1-4, when the sample 1 and the sample 2 were calculated for a relative ratio (Index) of the amount of the ADAMTS5 mRNA to the amount of the β-actin mRNA in the two kinds of healthy human's cDNAs (sample 1 and sample 2), the relative ratio (Index) in the sample 1 was 2.14 Index and the relative ratio (Index) in the sample 2 was 3.41 Index.

### Test Example 2: Quantification Through Real-Time PCR

Commercially available primer pair and probe (MGB probe) were used to perform the real-time PCR in the Test Examples 2-1 to 2-4.

### Test Example 2-1

The cDNA of the ADAMTS5 gene prepared in the Preparation Example 1 was diluted so as to be 1.0 μg/mL, 1.0×10⁻³ μg/mL, or 1.0×10⁻⁶ μg/mL to be used as a control DNA.

As the primer pair and the probe, the primer pair for the ADAMTS5 gene and the probe set available from Thermo Fisher Scientific K.K. (catalog number Hs00199841_m1; the probe is MGB probe; the sequences of the primer and the probe are not open to the public) were used.

The real-time PCR was performed using ABI PRISM 7000 (available from Thermo Fisher Scientific K.K.) and reaction reagents described in Table 2-1 under the below-described reaction conditions of the reaction time.

TABLE 2-1
Reaction reagents

| | | Final |
|---|---|---|
| | Amount | concentration |
| Universal Master Mix (2×) | 15 μL | 1× |
| (available from Thermo Fisher | | |
| Scientific K.K.) | | |
| Primer pair and probe set | 1.5 μL | |
| Control DNA | 3.0 μL | |
| Sterile distilled water | 10.5 μL | |
| Total | 30 μL | |

### —Reaction Time—

The real-time PCR was performed in the following order.

(1) 50° C. for 30 seconds

(2) 95° C. for 10 minutes

(3) The number of cycles: 50

Conditions: 95° C. for 15 seconds, then 60° C. for 60 seconds

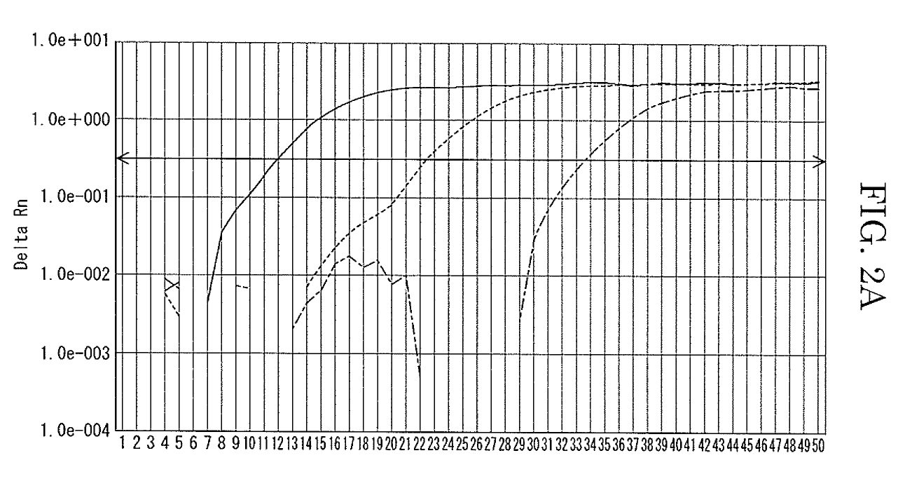

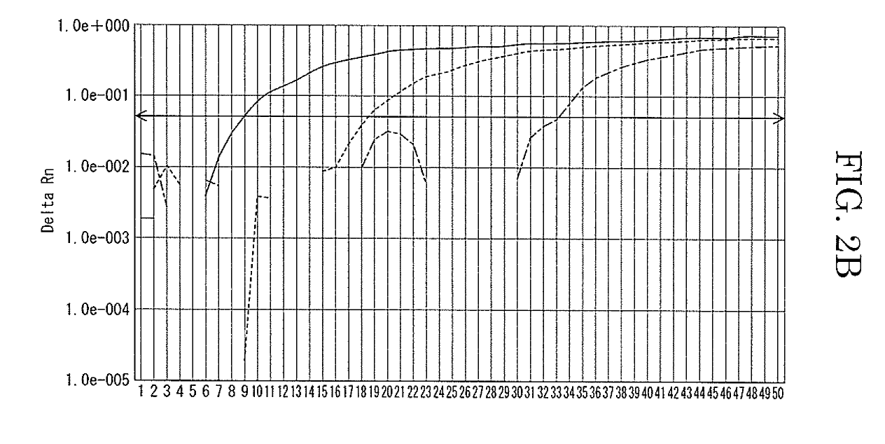

Amplification curves obtained through the real-time PCR are presented in FIG. 2A (horizontal axis: the number of cycles, longitudinal axis: fluorescent signal (ΔRn) corresponding to the amplified PCR products). In FIG. 2A, a solid line presents a result obtained when the control DNA of 1.0 μg/mL was used, a dotted line presents a result obtained when the control DNA of 1.0×10⁻³ μg/mL was used, a dot-dash line presents a result obtained when the control DNA of 1.0×10⁻⁶ μg/mL was used, and a line ends of which are arrows presents a base line (Threshold Line).

From the results of the amplification curves, a calibration curve was prepared with the longitudinal axis presenting a concentration of the control DNA and the horizontal axis presenting a Ct value (the number of cycles corresponding to Threshold).

### Test Example 2-2

Two kinds of healthy human's cDNAs (sample 1 and sample 2) were provided and were 2-fold diluted to prepare samples.

As the primer pair and the probe, the primer pair for the ADAMTS5 gene and the probe set (catalog number Hs00199841_m1; the probe is MGB probe; the sequences of the primer and the probe are not open to the public) available from Thermo Fisher Scientific K.K. were used in the same manner as in the Test Example 2-1.

The real-time PCR was performed under the same reaction conditions of the real-time PCR as in the Test Example 2-1 except that the control DNA in the Test Example 2-1 was changed to the sample 1 or 2.

When the calibration curve obtained in the Test Example 2-1 was used to calculate the samples 1 and 2 for an absolute amount of the ADAMTS5 gene, the result of the sample 1 was 5.86×10⁻⁴ μg/mL and the result of the sample 2 was 9.80×10⁻⁴ μg/mL.

### Test Example 2-3

The cDNA of the β-actin gene prepared in the Preparation Example 3 was diluted so as to be 1.0 μg/mL, 1.0×10⁻³ μg/mL, or 1.0×10⁻⁶ μg/mL to be used as a control DNA.

As the primer pair and the probe, the primer pair for the β-actin gene and the probe set available from Thermo Fisher Scientific K.K. (catalog number 4333762F; the probe is MGB probe; the sequences of the primer and the probe are not open to the public) were used.

The real-time PCR was performed using ABI PRISM 7000 (available from Thermo Fisher Scientific K.K.) and reaction reagents described in Table 2-2 under the below-described reaction conditions of the reaction time.

TABLE 2-2
Reaction reagents

| | | Final |
|---|---|---|
| | Amount | concentration |
| Universal Master Mix (2×) | 15 μL | 1× |
| (available from Thermo Fisher | | |
| Scientific K.K.) | | |
| Primer pair and probe set | 1.5 μL | |
| Control DNA | 3.0 μL | |
| Sterile distilled water | 10.5 μL | |
| Total | 30 μL | |

### —Reaction Time—

The real-time PCR was performed in the following order.

(1) 50° C. for 30 seconds

(2) 95° C. for 10 minutes

(3) The number of cycles: 50

Conditions: 95° C. for 15 seconds, then 60° C. for 60 seconds

Amplification curves obtained through the real-time PCR are presented in FIG. 2B (horizontal axis: the number of cycles, longitudinal axis: fluorescent signal (ΔRn) corresponding to the amplified PCR products). In FIG. 2B, a solid line presents a result obtained when the control DNA of 1.0 μg/mL was used, a dotted line presents a result obtained when the control DNA of 1.0×10⁻³ μg/mL was used, a dot-dash line presents a result obtained when the control DNA of 1.0×10⁻⁶ g/mL was used, and a line ends of which are arrows presents a base line (Threshold Line).

From the results of the amplification curves, a calibration curve was prepared with the longitudinal axis presenting a concentration of the control DNA and the horizontal axis presenting a Ct value (the number of cycles corresponding to Threshold).

### Test Example 2-4

Two kinds of healthy human's cDNAs (sample 1 and sample 2) were provided and were 50-fold diluted to prepare samples.

As a primer pair and a probe, the primer pair for the β-actin gene and the probe set (catalog number 4333762F; the probe is MGB probe; the sequences of the primer and the probe are not open to the public) available from Thermo Fisher Scientific K.K. were used in the same manner as in the Test Example 2-3.

The real-time PCR was performed under the same reaction conditions of the real-time PCR as in the Test Example 2-3 except that the control DNA in the Test Example 2-3 was changed to the sample 1 or 2.

When the calibration curve obtained in the Test Example 2-3 was used to calculate the samples 1 and 2 for an absolute amount of the β-actin gene, the result of the sample 1 was 2.12 μg/mL and the result of the sample 2 was 2.74 μg/mL.

From the results of Test Examples 2-1 to 2-4, when the sample 1 and the sample 2 were calculated for a relative ratio (Index) of the amount of the ADAMTS5 mRNA to the amount of the β-actin mRNA in the two kinds of healthy human's cDNAs (sample 1 and sample 2), the relative ratio (Index) in the sample 1 was 2.77 Index and the relative ratio (Index) in the sample 2 was 3.58 Index.

Results of Test Examples 1 and 2 are summarized in Table 3.

TABLE 3

| | | ADAMTS5 | | | β-actin | | | | |
|---|---|---|---|---|---|---|---|---|---|
| | | | | Absolute | | | Absolute | Ratio | |
| Sample | Primer pair/ | Ct | Dilution | amount | Ct | Dilution | amount | ADAMTS5/ | |
| name | probe | value | ratio | (μg/mL) | value | ratio | (μg/mL) | β-actin | Index |
| Sample 1 | Commercially | 33.82 | 2 | 5.86 × 10⁻⁴ | 25.74 | 50 | 2.12 | 2.77 × 10⁻⁴ | 2.77 |
| | available | | | | | | | | |
| | product | | | | | | | | |
| | Present | 33.23 | 2 | 1.07 × 10⁻³ | 26.00 | 50 | 5.00 | 2.14 × 10⁻⁴ | 2.14 |
| | invention | | | | | | | | |
| Sample 2 | Commercially | 33.00 | 2 | 9.80 × 10⁻⁴ | 25.28 | 50 | 2.74 | 3.58 × 10⁻⁴ | 3.58 |
| | available | | | | | | | | |
| | product | | | | | | | | |
| | Present | 32.16 | 2 | 2.18 × 10⁻³ | 25.85 | 50 | 6.40 | 3.41 × 10⁻⁴ | 3.41 |
| | invention | | | | | | | | |

From the results of Test Examples 1 and 2, an expression level (Index value) (Test Example 1) of the ADAMTS5 gene in the healthy human's cDNA quantified through the real-time PCR by using the combination of the primer pair and the probe of the present invention is substantially the same expression level as an expression level (Index value) (Test Example 2) of the ADAMTS5 gene in the healthy human's cDNA quantified through the real-time PCR by using the commercially available MGB probe.

Therefore, it was indicated that use of the combination of the primer pair and the probe of the present invention was able to quantify a relative ratio of the amount of the ADAMTS5 mRNA to the amount of the β-actin mRNA at the lower cost and the same level compared to use of the conventional, costly MGB probe.

### Test Example 3: Comparison of Performance of Real-Time PCR

The primer pair for the ADAMTS5 gene (SEQ ID NOs: 2 and 3) and the probe (SEQ ID NO: 4) prepared in the Preparation Example 2, and the commercially available primer pair for the ADAMTS5 gene and the probe set (available from Thermo Fisher Scientific K.K., catalog number Hs00199841_m1; the probe is the MGB probe; the sequences of the primer and the probe are not open to the public) were compared for performances of the real-time PCR in the following manners.

### Test Example 3-1: Primer Pair and Probe of the Present Invention

The real-time PCR was performed in the same manner as in the Test Example 1-1. The control DNA having each concentration was subjected to the real-time PCR five times to determine each Ct value. Results are presented in Table 4.

### Test Example 3-2: Commercially Available Primer Pair and Probe

The real-time PCR was performed in the same manner as in the Test Example 2-1. The control DNA having each concentration was subjected to the real-time PCR five times to determine each Ct value. Results are presented in Table 4.

TABLE 4

| Primer pair | | | | | | |
|---|---|---|---|---|---|---|
| and probe for | Commercially | | | | | |
| ADAMTS5 | available product | | | Present invention | | |
| Control DNA | (Test Example 3-2) | | | (Test Example 3-1) | | |
| concentration | | 1.0 × | 1.0 × | | 1.0 × | 1.0 × |
| (μg/mL) | 1.0 | 10⁻³ | 10⁻⁶ | 1.0 | 10⁻³ | 10⁻⁶ |
| Ct value (1st) | 11.53 | 26.61 | 35.76 | 11.83 | 26.24 | 34.00 |
| Ct value (2nd) | 10.74 | 26.00 | 36.73 | 11.75 | 24.84 | 33.06 |
| Ct value (3rd) | 9.89 | 26.54 | 36.28 | 9.66 | 24.67 | 33.00 |
| Ct value (4th) | 10.26 | 22.16 | 33.00 | 10.04 | 19.39 | 29.36 |
| Ct value (5th) | 10.43 | 19.71 | 32.44 | 11.39 | 22.16 | 32.16 |
| Average value | 10.57 | 24.20 | 34.84 | 10.93 | 23.46 | 32.32** |
| of Ct values | | | | | | |

**p < 0.01 (Compared to the commercially available product)

From the results of the Test Example 3, when the control DNA having a low concentration (1.0×10⁻⁶ μg/mL) was a sample DNA, the Ct value obtained when the combination of the primer pair and the probe of the present invention was used (Test Example 3-1: 32.32) was significantly lower (p<0.01) than the Ct value obtained when the combination of the commercially available primer pair and probe (Test Example 3-2: 34.84) was used.

Therefore, it was indicated that combination of the primer pair and the probe of the present invention could detect an absolute amount of the amount of the ADAMTS5 mRNA at higher sensitivity compared to use of the conventional MGB probe.

Aspects of the present invention are as follows, for example.

<1> A method for quantifying an ADAMTS5 gene, the method including:

measuring an amount of the ADAMTS5 gene in a sample through a quantitative PCR,

wherein a primer consisting of a nucleotide sequence set forth in SEQ ID NO: 2 below and a primer consisting of a nucleotide sequence set forth in SEQ ID NO: 3 below are used as an amplification primer pair for amplifying a region including the ADAMTS5 gene,

(SEQ ID NO: 2)
5′-CTAAGCCCTGGTCCAAATGC-3′;
and
(SEQ ID NO: 3)
5′-CCAGCAAACAGTTACCATGGC-3′.

<2> The method for quantifying the ADAMTS5 gene according to <1>, wherein a probe consisting of a nucleotide sequence set forth in SEQ ID NO: 4 below is used as a detection probe for the ADAMTS5 gene,

(SEQ ID NO: 4)
5′-TTCAGCCACCATCACAGAATTCCTGGA-3′.

<3> The method for quantifying the ADAMTS5 gene according to <1> or <2>, further including measuring an amount of a β-actin gene in the sample through the quantitative PCR,

wherein a primer consisting of a nucleotide sequence set forth in SEQ ID NO: 6 below and a primer consisting of a nucleotide sequence set forth in SEQ ID NO: 7 below are used as an amplification primer pair for amplifying a region including the β-actin gene,

(SEQ ID NO: 6)
5′-CCAGCTCACCATGGATGATG-3′;
and
(SEQ ID NO: 7)
5′-CCTTGCACATGCCGGAG-3′.

<4> The method for quantifying the ADAMTS5 gene according to <3>, wherein a probe consisting of a nucleotide sequence set forth in SEQ ID NO: 8 below is used as a detection probe for the β-actin gene,

(SEQ ID NO: 8)
5′-CCGCGCTCGTCGTCGACAAC-3′.

<5> The method for quantifying the ADAMTS5 gene according to any one of <1> to <4>, wherein the amount of the ADAMTS5 gene to be quantified is at least one of an absolute amount of the ADAMTS5 gene and a relative amount of the ADAMTS5 gene to an amount of the β-actin gene.
<6> A primer pair used for quantifying an ADAMTS5 gene in a sample through a quantitative PCR, the primer pair consisting of.

a primer consisting of a nucleotide sequence set forth in SEQ ID NO: 2 below; and

a primer consisting of a nucleotide sequence set forth in SEQ ID NO: 3 below,

(SEQ ID NO: 2)
5′-CTAAGCCCTGGTCCAAATGC-3′;
and
(SEQ ID NO: 3)
5′-CCAGCAAACAGTTACCATGGC-3′.

<7> A combination of a primer pair and a probe used for quantifying an ADAMTS5 gene in a sample through a quantitative PCR,

wherein the primer pair is the primer pair according to <6>, and

wherein the probe is a probe consisting of a nucleotide sequence set forth in SEQ ID NO: 4 below:

(SEQ ID NO: 4)
5′-TTCAGCCACCATCACAGAATTCCTGGA-3′.

<8> A kit used for quantifying an ADAMTS5 gene in a sample through a quantitative PCR, the kit including:

the primer pair according to <6>.

<9> The kit according to <8>, further including a probe consisting of a nucleotide sequence set forth in SEQ ID NO: 4 below as a detection probe for the ADAMTS5 gene,

(SEQ ID NO: 4)
5′-TTCAGCCACCATCACAGAATTCCTGGA-3′.

<10> The kit according to <8> or <9>, further including a primer consisting of a nucleotide sequence set forth in SEQ ID NO: 6 below and a primer consisting of a nucleotide sequence set forth in SEQ ID NO: 7 below as an amplification primer pair for amplifying a region including a β-actin gene,

(SEQ ID NO: 6)
5′-CCAGCTCACCATGGATGATG-3′;
and
(SEQ ID NO: 7)
5′-CCTTGCACATGCCGGAG-3′.

<11> The kit according to any one of <8> to <10>, further including a probe consisting of a nucleotide sequence set forth in SEQ ID NO: 8 below as a detection probe for the β-actin gene,

(SEQ ID NO: 8)
5′-CCGCGCTCGTCGTCGACAAC-3′.

### INDUSTRIAL APPLICABILITY

According to the method of the present invention for quantifying the ADAMTS5 gene, it is possible to quantify the ADAMTS5 gene in a sample at the lower cost at higher sensitivity. Therefore, the method of the present invention can be suitably used for predicting drug efficacy that uses the amount of the ADAMTS5 gene in the sample as an indicator.

The primer pair, the combination of the primer pair and the probe, and the kit of the present invention can be suitably used for the method of the present invention for quantifying the ADAMTS5 gene.

Examples of the prediction of drug efficacy that uses the amount of the ADAMTS5 gene in the sample as an indicator include a method for evaluating whether an anti-TNFα antibody drug is effective to a rheumatoid arthritis.

More specifically, when an expression level of the mRNA of ADAMTS5 (expression level of mRNA of ADAMTS5/expression level of mRNA of β-actin) is less than 0.6× 1/25 in the case where β-actin is used as an endogenous control, it is possible to evaluate that Infliximab is effective to rheumatoid arthritis. In addition, when an expression level of the mRNA of ADAMTS5 (expression level of mRNA of ADAMTS5/expression level of mRNA of β-actin) is 4.0×10⁻⁴ or more in the case where β-actin is used as an endogenous control, it is possible to evaluate that Adalimumab is effective to rheumatoid arthritis.

1. A method for quantifying an ADAMTS5 gene, the method comprising:
measuring an amount of the ADAMTS5 gene in a sample through a quantitative PCR,
wherein a primer consisting of a nucleotide sequence set forth in SEQ ID NO: 2 below and a primer consisting of a nucleotide sequence set forth in SEQ ID NO: 3 below are used as an amplification primer pair for amplifying a region including the ADAMTS5 gene,
(SEQ ID NO: 2)
5′-CTAAGCCCTGGTCCAAATGC-3′;
and
(SEQ ID NO: 3)
5′-CCAGCAAACAGTTACCATGGC-3′.

2. The method for quantifying the ADAMTS5 gene according to claim 1, wherein a probe consisting of a nucleotide sequence set forth in SEQ ID NO: 4 below is used as a detection probe for the ADAMTS5 gene,
(SEQ ID NO: 4)
5′-TTCAGCCACCATCACAGAATTCCTGGA-3′.

3. The method for quantifying the ADAMTS5 gene according to claim 1 or 2, further comprising measuring an amount of a β-actin gene in the sample through the quantitative PCR,
wherein a primer consisting of a nucleotide sequence set forth in SEQ ID NO: 6 below and a primer consisting of a nucleotide sequence set forth in SEQ ID NO: 7 below are used as an amplification primer pair for amplifying a region including the β-actin gene,
(SEQ ID NO: 6)
5′-CCAGCTCACCATGGATGATG-3′;
and
(SEQ ID NO: 7)
5′-CCTTGCACATGCCGGAG-3′.

4. The method for quantifying the ADAMTS5 gene according to claim 3, wherein a probe consisting of a nucleotide sequence set forth in SEQ ID NO: 8 below is used as a detection probe for the β-actin gene,
(SEQ ID NO: 8)
5′-CCGCGCTCGTCGTCGACAAC-3′.

5. The method for quantifying the ADAMTS5 gene according to any one of claims 1 to 4, wherein the amount of the ADAMTS5 gene to be quantified is at least one of an absolute amount of the ADAMTS5 gene and a relative amount of the ADAMTS5 gene to an amount of the β-actin gene.

6. A primer pair used for quantifying an ADAMTS5 gene in a sample through a quantitative PCR, the primer pair consisting of:
a primer consisting of a nucleotide sequence set forth in SEQ ID NO: 2 below; and
a primer consisting of a nucleotide sequence set forth in SEQ ID NO: 3 below,
(SEQ ID NO: 2)
5′-CTAAGCCCTGGTCCAAATGC-3′;
and
(SEQ ID NO: 3)
5′-CCAGCAAACAGTTACCATGGC-3′.

7. A combination of a primer pair and a probe used for quantifying an ADAMTS5 gene in a sample through a quantitative PCR,
wherein the primer pair is the primer pair according to claim 6, and
wherein the probe is a probe consisting of a nucleotide sequence set forth in SEQ ID NO: 4 below:
(SEQ ID NO: 4)
5′-TTCAGCCACCATCACAGAATTCCTGGA-3′.

8. A kit used for quantifying an ADAMTS5 gene in a sample through a quantitative PCR, the kit comprising:
the primer pair according to claim 6.

9. The kit according to claim 8, further comprising a probe consisting of a nucleotide sequence set forth in SEQ ID NO: 4 below as a detection probe for the ADAMTS5 gene,
(SEQ ID NO: 4)
5′-TTCAGCCACCATCACAGAATTCCTGGA-3′.

10. The kit according to claim 8 or 9, further comprising a primer consisting of a nucleotide sequence set forth in SEQ ID NO: 6 below and a primer consisting of a nucleotide sequence set forth in SEQ ID NO: 7 below as an amplification primer pair for amplifying a region including a β-actin gene,
(SEQ ID NO: 6)
5′-CCAGCTCACCATGGATGATG-3′;
and
(SEQ ID NO: 7)
5′-CCTTGCACATGCCGGAG-3′.

11. The kit according to any one of claims 8 to 10, further comprising a probe consisting of a nucleotide sequence set forth in SEQ ID NO: 8 below as a detection probe for the β-actin gene,
(SEQ ID NO: 8)
5′-CCGCGCTCGTCGTCGACAAC-3′.
